(19) United States
(12) Patent Application Publication
(10) Pub. No.: US 2011/0214195 A1
(43) Pub. Date: Sep. 1, 2011

(54) Methods For Treating Clinical Conditions Associated With Lipoprotein Lipase Activity

(75) Inventors: Robert H. Eckel, Morrison, CO (US); Hong Wang, Centennial, CO (US)

(73) Assignee: The Regents of the University of Colorado, a body corporate, Denver, CO (US)

(21) Appl. No.: 13/127,695

(22) PCT Filed: Nov. 9, 2009
(86) PCT No.: PCT/US2009/063743

§ 371 Date: May 4, 2011

Related U.S. Application Data
(60) Provisional application No. 61/113,135, filed on Nov. 10, 2008.

(51) Int. Cl. A01K 67/027; C12Q 1/44; A61K 38/46; A61K 31/713

(52) U.S. Cl. 800/18; 435/19; 424/94.6; 514/44 A; 800/14

(57) ABSTRACT

The present invention provides methods for treating a clinical condition associated with lipoprotein lipase activity in the brain of a subject.

### FIELD OF THE INVENTION

The present invention relates to treating a clinical condition associated with lipoprotein lipase activity in the brain of a subject.

### BACKGROUND OF THE INVENTION

While the role of LPL in various cells has previously been recognized, currently there has been no known report regarding the role of LPL in the brain.

The present inventors have discovered that a selective knockout of the lipoprotein lipase (LPL) gene in the brain leads to development of severe obesity in mammals. The present inventors have also determined that the effect is mediated by the hippocampus. Since the hippocampus is involved in memory function, it is believed that LPL also plays role in other diseases such as memory related diseases, for example, Alzheimer's disease, age related senility, as well as other memory related diseases.

Accordingly, there is a need for modulating the level of LPL in the brain to treat various clinical conditions.

### SUMMARY OF THE INVENTION

Various methods and compositions for treating or preventing a clinical condition associated with lipoprotein lipase activity in the brain of a subject is provided. Some aspects of the invention comprise modulating the LPL activity in the brain of the subject. In some embodiments, such a modulation is accomplished by administering to the subject a composition comprising a compound that modulates lipoprotein lipase activity in the brain of the subject. Within these embodiments, in some instances the compound modulates the expression of lipoprotein lipase in the brain of the subject. In some cases, the compound increases the expression of lipoprotein lipase in the brain of the subject.

Generally, any clinical condition associated with the LPL activity in the brain can be treated or prevented using methods and/or compositions of the invention. In some particular embodiments, the clinical condition comprises obesity, Alzheimer's disease, dementia, memory loss, or depression.

Typically, the compound increases the lipoprotein lipase activity or the expression of LPL in the brain of the subject.

In some embodiments, the compound is LPL or an analog or a derivative thereof. When LPL is used in methods of the invention, it can be enzymatically active or enzymatically inactive.

Still in other embodiments, the compound upregulates the enzymatic activity of LPL in the brain. In other embodiments, the compound downregulates the enzymatic activity of LPL in the brain. Yet in other embodiments, the compound upregulates the expression of LPL in the brain. Still yet in other embodiments, the compound down-regulates the expression of LPL in the brain. One particular compound for modulating the expression of LPL in the brain include, but are not limited to, small or short interfering RNAs (siRNAs). Such siRNAs typically modulate translation of mRNA. Compounds of the invention, however, include those that also modulate transcription of LPL gene in the brain.

Other aspects of the invention provide a mouse model to identify biochemical and cellular mechanisms relating to LPL activity in the brain. The mouse comprises NEXLPL−/− genotype. Such mice can be produced from Nex-Cre mice to generate brain-specific LPL knockout mice using the crelox-P recombination system.

Yet other aspects of the invention provide various transgenic non-human mammals comprising neuronal specific LPL deficiency compared to the wild type (or control) of said non-human mammal. It should be appreciated that the wild type or control refers to a non-human mammal having a normal neuronal specific LPL activity level. A variety of non-human mammals can be produced by methods disclosed herein, for example, canine, equine, pig, mouse, cattle, simian, etc. In some embodiments, the mammal is a rodent. Within these embodiments, in some instances the rodent is a mouse. Typically, the phenotype of the mouse is NEXLPL−/− or NEXLPL+/−.

Still other aspects of the invention provide methods for identifying a small molecule that is suitable for treating or preventing a clinical condition associated with decreased lipoprotein lipase activity in the brain of a subject. Typically, such methods comprise:
administering the small molecule to a non-human mammal comprising neuronal specific LPL deficiency compared to the wild type of said non-human mammal; and

determining the level of neuronal specific LPL activity in the non-human mammal, wherein if the level of neuronal specific LPL activity in the non-human mammal is substantially similar to the level of neuronal specific LPL activity in the control, then it is indication that the small molecule is suitable for treating or preventing clinical condition associated with decreased lipoprotein lipase activity in the brain of a subject. Typically, the subject is human.

### BRIEF DESCRIPTION OF THE DRAWINGS

FIG. 1A is a graph showing lipoprotein lipase mRNA levels in different brain regions between wild type (WT) and NEXLPL−/− mice.

FIG. 1B is a graph showing LPL activity in peripheral tissues. Heparin-releasable LPL activities were measured in heart, WAT, skeletal muscle, and BAT. LPL activity was expressed as nanomoles of FFA per minute per gram tissue.

FIG. 1C is a graph showing differences between WT and NEXLPL−/− mice body weight for both male and female.

FIG. 1D is a graph showing body composition differences between wild type (WT) and NEXLPL−/− mice.

FIG. 1E is a graph showing the plasma insulin level difference between WT and NEXLPL−/− mice.

FIG. 1F is a graph showing the plasma leptin level difference between WT and NEXLPL−/− mice.

FIG. 2A is a graph showing the difference between WT and NEXLPL−/− mice of average daily food intake.

FIG. 2B is a graph showing the difference between WT and NEXLPL−/− mice in average respiratory quotient (RQ).

FIG. 2C is a graph showing the difference between WT and NEXLPL−/− mice in average metabolic rate (MR).

FIG. 2D is a graph showing the difference between WT and NEXLPL−/− mice in average physical activity (insert: average total physical activity).

FIG. 2E is a graph showing the difference of body weights of NEXLPL+/− mice at 6 and 12 mo.

FIG. 2F is a graph showing the difference of fat mass percentages of NEXLPL+/− mice at 6 and 12 mo.

FIG. 2G is a graph showing correlation between average daily physical activities vs. body fat mass percentage for NEXLPL+/− mice at 12 mo.

FIG. 2H is a graph showing weight gain at 46 wk vs. food intake at the 30^{th} week for NEXLPL+/− mice.

FIG. 3A is a graph showing the difference in average daily food intake between WT and NEXLPL+/− mice at 6 mo.

FIG. 3B is a graph showing the difference of average RQ between WT and NEXLPL+/− mice at 6 and 12 mo.

FIG. 3C is a graph showing the difference of average MR between WT and NEXLPL+/− mice at 6 and 12 mo.

FIG. 3D is a graph showing the difference of average total physical activity between WT and NEXLPL+/− mice at 6 and 12 mo.

FIG. 4A is a graph of gene expression in the hypothalamus of NEXLPL−/− vs. WT.

FIG. 4B is a graph of gene expression in the hippocampus of NEXLPL−/− vs. WT.

FIG. 4C is a graph of gene expression of the Mc4/3r pathway genes in the hypothalamus of WT, NEXLPL−/− and NEXLPL+/−.

FIG. 5A is a graph showing four major classes of lipids in hypothalamus area of in WT, NEXLPL+/− and NEXLPL−/− mice at 12 mo.

FIG. 5B is a graph showing distribution of different TAG species in hypothalamus area (Insert: SCD-1 gene expression in hypothalamus) of WT, NEXLPL+/− and NEXLPL−/− mice at 12 mo.

FIG. 5C is a graph showing distribution of different FFA species in hypothalamus area of WT, NEXLPL+/− and NEXLPL−/− mice at 12 mo.

FIG. 5D is a graph showing relative lipid concentrations of n-3 and n-6 FFAs in hypothalamus area of WT, NEXLPL+/− and NEXLPL−/− mice at 12 mo.

FIG. 5E is a graph showing relative lipid concentration for TAG (18:0/16:0/20:4) in hypothalamus area of WT, NEXLPL+/− and NEXLPL−/− mice at 12 mo.

FIG. 5F is a graph showing relative lipid concentration for DAG (18:0/20/4) in hypothalamus area of WT, NEXLPL+/− and NEXLPL−/− mice at 12 mo.

FIG. 5G is a graph showing relative lipid concentration for MAG (20:4) in hypothalamus area of WT, NEXLPL+/− and NEXLPL−/− mice at 12 mo.

FIG. 5H is a graph showing relative lipid concentration for FFA (20:4) in hypothalamus area of WT, NEXLPL+/− and NEXLPL−/− mice at 12 mo.

FIG. 5I is a graph showing desaturase gene expression levels in hypothalamus of WT, NEXLPL+/− and NEXLPL−/− mice at 12 mo.

FIG. 5J is a graph showing desaturase gene expression levels in liver of WT, NEXLPL+/− and NEXLPL−/− mice at 12 mo.

FIG. 6 is a schematic illustration of LPL-mediated neutral lipid uptake; and

FIGS. 7A and 7B show the hippocampus and mediobasal hypothalamus (MBH) area of injection, respectively, for the lentivirus co-expressing GFP and LPL and the two controls into of adult (3 months old) female NEXLPL−/− mice.

### DETAILED DESCRIPTION OF THE INVENTION

Lipoprotein lipase (LPL) is a multifunctional enzyme produced by and studied in many tissues including adipose tissue and muscle. Following synthesis in parenchymal cells, the enzyme protein is transported to the endothelium where it binds to the glycocalyx. Here, the lipase is rate-limiting for the hydrolysis of the triglyceride core of the circulating triglyceride-rich lipoproteins, chylomicrons and very low density lipoproteins (VLDL). The reaction products, fatty acids and monoacylglycerol, are in part taken up by the tissues locally where they are processed in a tissue-specific manner, e.g., stored as neutral lipids (triglycerides>>cholesteryl esters) in adipose tissue, or oxidized or stored in muscle. Exemplary of the tissue-specific effects of LPL are skeletal muscle specific transgenic mice that are insulin resistant and relatively lean, and skeletal muscle specific LPL knockout mice that have increases in skeletal muscle insulin-mediated glucose uptake and subsequently develop obesity. Once LPL is bound to lipoproteins, in some tissues, e.g., liver, muscle, and macrophages, both the active and inactive lipase protein have also been shown to enhance the binding, uptake and degradation of lipoproteins by mechanisms that are both lipoprotein receptor-dependent and independent.

LPL is also present throughout the nervous system, including the brain, spinal cord, and peripheral nerve. In the brain, LPL mRNA is found in neurons in the dentate gyrus as well as the CA1-CA4 layers in the hippocampus, pyramidal cells in the cortex, and the Purkinje cells in the cerebellum; and, in experiments using ³⁵S methionine, LPL protein is synthesized in the brain. Although the mRNA for LPL is in neurons, the lipase protein is distributed on endothelial surfaces throughout the brain. This discrepancy implicates the binding of circulating LPL protein (active and/or inactive) to the endothelium.

It is interesting to note that patients with LPL deficiency in the brain appears to have neuropsychiatric findings including dementia, memory loss, and depression. The mild nature of these symptoms, however, may relate to the fact that nearly all LPL deficiency syndromes are in patients who are compound heterozygotes inhabiting missense mutations wherein the enzyme protein is present but hydrolytically inactive. Although LPL itself has been localized in the senile plaque, the relationship of LPL mutations to the components of the chylomicronemia syndrome including the dementia has been conflicting. It is important to consider, however, that non-hydrolytic functions of the lipase could still prevail in the setting of LPL deficiency and severe hypertriglyceridemia including effects in the CNS.

The present inventors have discovered that LPL is also present in the lower spinal cord and cauda equina with much of the labeling in the spinal cord seen in white matter. In addition, the present inventors have shown that LPL-mediated hydrolysis of exogenous triacylglycerol is an important source of free fatty acid (FFA) for cultured Schwann cells, and thus it is believed that the enzyme plays a role in myelin biosynthesis in the peripheral nervous system.

The transport of fatty acids into the brain is mediated by the uptake of albumin-bound FFA or from lipoproteins by LPL. Therefore, LPL is present in brain capillaries and mediate such a function. Evidence exists that inflammation increases the entry of lipids into the CNS, and that fatty acids can be used to assess the integrity of the blood brain barrier in humans. The fact that synthesis of fatty acids does not correlate well with the accumulation of phospholipid fatty acids in myelin indicates exogenous uptake mechanisms. In addition, although the major saturated and monounsaturated fatty acids are accounted for by de novo synthesis within the developing rat brain, the content of the essential n-6 and n-3 fatty acids in the brain is dependent on transport. This transport and incorporation of plasma polyunsaturated fatty acids into the nervous system continues throughout life. In the rat brain, recent estimates are that at least 3-5% of esterified brain arachidonic acid and 2-8% of brain docosahexaenoic acid (DHA) are replaced daily by dietary n-6 and n-3 fatty acids, respectively. These rates predict half-lives of 1-2 wk for plasma-brain exchange of arachidonic acid and DHA. And, when a simple irreversible uptake model derived from rat studies was modified for positron emission tomography (PET) and applied to adult humans to quantify the incorporation of ¹¹C arachidonic acid uptake into the brain, rates were 5.6±1.2 and 2.6±0.5 μL/min/ml in gray and white matter, respectively.

In the neuron, a number of functions of LPL have been suggested. These include the ability of active LPL to promote the phenotypical differentiation of cultured neuroblastoma cells, a function of LPL that is dependent on the active lipase not the inactive protein. Moreover, in these same cells LPL prevented neurotoxicity when cells were exposed to native or oxidized lipoproteins. These observations are particularly relevant in that the dentate gyrus, one of the regions of the hippocampus in which LPL mRNA is localized, is one of the few regions of the adult brain that undergoes neurogenesis. These in vitro data show that LPL plays a role in the differentiation of neurons in vivo and in the response to oxidative stress in several neurodegenerative disorders. Supportive in vivo evidence of the protective role of LPL in the brain was an increase in LPL mRNA and protein in the hippocampus two days after traumatic brain injury. Another potential in vivo role of LPL comes from studies using a porcine in vitro blood-brain barrier model wherein LPL facilitated the uptake and transcytosis of LDL and LDL-associated α-tocopherol.

It appears that patients with LPL deficiency have neuropsychiatric findings including dementia, memory loss, and depression. The mild nature of these symptoms, however, may relate to the fact that nearly all LPL deficiency syndromes are in patients who are compound heterozygotes inhabiting missense mutations wherein the enzyme protein is present but hydrolytically inactive. Although LPL itself has been localized in the senile plaque, the relationship of LPL mutations to the components of the chylomicronemia syndrome including the dementia has been conflicting. Non-hydrolytic functions of the lipase could still prevail in the setting of LPL deficiency and severe hypertriglyceridemia including effects in the CNS.

The expression of LPL mRNA occurs throughout the brain, but most impressively in the CA1-CA4 layers of the hippocampus. This area is well recognized to be the “learning center” of the brain, where hippocampal neurons contribute to memory by rapidly assimilating information about the perceptual and behavioral structure of experience. The process by which this occurs is called long term potentiation (LTP) whereby a series of conditioned impulses potentiate the size of synaptic potentials. It appears that the hippocampus is also involved in body weight regulation. One potential mediator of this process is brain-derived neurotrophic factor (BDNF) that is not only synthesized in the hippocampus but known to play an important role in activity-dependent synaptic plasticity in the adult brain. In rodents, administration of exogenous BDNF unilaterally or bilaterally into the ventromedial hypothalamus decreases food intake, whereas genetic models with deficient BDNF signaling or viral-mediated selective knock-down of BDNF in the ventromedial and dorsomedial nuclei of the hypothalamus exhibit hyperphagia and obesity. In addition, impaired performance on hippocampal-dependent learning and memory tasks and reduced hippocampal BDNF is also found in rats that have been maintained on a high-fat diet. Moreover, exercise increases hippocampal BDNF mRNA and protein, and particularly in rats that were already familiarized to the exercise regimen. Of interest, obese children and adolescents have reduced serum BDNF levels. Furthermore, a recent report puts BDNF into an even greater perspective in humans. Wilms' tumor, aniridia, genitourinary anomalies, and mental retardation (WAGR) syndrome is a heterozygous, variably sized, contiguous gene deletion causing haplo-insufficiency of the WT1 and PAX6 genes on chromosome 11p13, approximately 4 Mb centromeric to BDNF (11p14.1). Patients with heterozygous BDNF deficiency (58% of the WAGR registry) had higher BMI z scores throughout childhood than did patients with intact BDNF.

Current research and theory have in general considered the effects of BDNF on energy regulation and on cognitive functioning as largely independent phenomena involving distinct neural substrates, e.g., hypothalamic and hippocampal, respectively. Of relevance and interest, however, when a pseudorabies virus, that encodes a GFP marker and only replicates in neurons that express cre recombinase, was injected into the arcuate nucleus of the hypothalamus of mice that expressed cre only in neurons that express NPY or the leptin receptor, the retrograde tracing of dye revealed inputs into the arcuate nucleus from other areas of the hypothalamus, as well as the amygdala, cortex, and other brain regions including the CA1 region of the hippocampus. The present inventors have discovered that overall, the BDNF data and the synaptic connections between the hypothalamus and hippocampus indicate a process by which body weight and/or fat are regulated. Once achieved, weight loss is difficult to maintain and a biological process of “metabolic memory” seems plausible. Conceptually, this process could become hard-wired and operate in a manner by which an animal associates increases or decreases in food intake with other physiological changes in energy balance. The present inventors have found that in some instances such a process appears to be related to the presence of LPL in the hippocampus and/or hypothalamus. Moreover, the present inventors have shown that LPL in the brain is important to energy balance and body weight regulation.

In recent years a series of experiments have demonstrated that when fatty acids are delivered through central injections into the third ventricle, reductions in food intake occurred. Following the intracerebroventricular (ICV) administration of oleic acid, endogenous glucose production and food intake were markedly inhibited. It was also demonstrated that this effect was related to fatty acid oxidation in that the biochemical inhibition of hypothalamic CPT1 activity also diminished food intake and endogenous glucose production. However, after three days of overfeeding this effect of oleic acid was obliterated. Subsequent studies showed that when CPT1 activity was inhibited in the setting of overfeeding hypothalamic levels of LCFA-CoAs were normalized and food intake and hepatic glucose production were again inhibited. Although these experiments seem to indicate that the central inhibition of lipid oxidation is sufficient to restore hypothalamic lipid sensing as well as glucose metabolism and energy homeostasis, very recent experiments have demonstrated that mice with a brain-specific knockout of CPT1c, a homologous form of CPT1 that binds malonyl-CoA but does not support fatty acid oxidation, exhibited decreased food intake and less weight gain on 10% fat but gained excessive weight on a 60% fat diet while maintaining lower or equivalent food intake. Thus, CPT1c also appears to play a role in energy balance and body weight regulation, but not by increasing fatty acid oxidation.

The inhibition of the rate limiting step of lipogenesis, fatty acid synthase (FAS), also decreases appetite and body weight over short intervals. In mice with a conditional combined pancreatic 0 cell and hypothalamic FAS deficiency a dramatic phenotype of leanness, hypophagia with increased physical activity and impaired hypothalamic PPARα signaling was noted for up to 6 months. However, when a PPARα agonist was administered into the hypothalamus PPARα target gene expression increased and food intake was normalized. Importantly, inactivation of β cell FAS enzyme activity had no noticeable effect on islet function in culture or in vivo. Overall, these experiments not only demonstrated the importance of lipogenesis in the hypothalamus in regulating food intake but also in regulating physical activity, an effect apparently mediated through ligands generated by FAS that induced PPARα gene expression.

Discovery by the present inventors that mice with LPL deficiency in NEX neurons develop obesity shows that two diametrically opposed proteins, one induced by feeding (FAS) and the other induced by starvation (PPARα) form an integrative sensory module in the central nervous system to determine energy balance. The absence of fatty acids to generate LCFA-CoAs from lipogenesis or from LPL deficiency appears to support a pathway of preferential fatty acid oxidation and weight gain.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Example 1

The present inventors have identified LPL in different brain regions including the cerebellum (Ce), cerebral cortex (Co), hypothalamus (Hy), pituitary (Pi), and hippocampus (Hi). In the hippocampus, LPL mRNA is widely distributed as visualized by in situ hybridization. It is believed that lipids are important regulators of energy balance and body weight/composition. The Nex-Cre mice were utilized to generate brain-specific LPL knockout mice using the crelox-P recombination system. See “Genetic targeting of principal neurons in neocortex and hippocampus of NEX-Cre mice,” Genesis, 2006, 44, 611-621, which is incorporated herein by reference in its entirety. Briefly, LPL loxP mice were crossed with transgenic mice with brain-specific expression of cre recombinase driven by the regulatory sequences of NEX, a gene that encodes a neuronal basic helix-loop-helix (bHLH) protein. Controlled breeding into different lines of lacZ-indicator mice demonstrated that cre activity was expressed in the neocortex, hippocampus, mid- and hindbrain regions, pyramidal neurons and dentate gyrus mossy and granule cells in the dorsal telencephalon, but was absent from proliferating neural precursors of the ventricular zone, interneurons, oligodendrocytes, and astrocytes.

Once generated, male and female NEXLPL−/−, NEXLPL+/− and wild type (WT) littermate WT mice were housed at ˜20° C. on a 12:12 h light-dark photoperiod and provided standard rodent chow and water ad libitum. Body weight was monitored on a weekly basis for individualized caged mice.

### LPL Activity Assay

Mice were anesthetized with an ip administration of Avertin (2,2,2-tribromoethanol, 250 mg/kg) after a 4 hr fast. Tissues were dissected and assayed immediately. Heparin-releasable LPL activity was measured in heart, WAT, skeletal muscle and BAT. LPL activity was expressed as nanomoles of FFA per minute per gram tissue.

### Measurement of Body Weight, Body Composition, and Plasma Metabolic Parameters

Body composition was measured on anesthetized mice by dual-energy x-ray absorptiometry using a mouse densitometer (PIXImus2, Lunar Corp., Madison, Wis.). Blood was collected by cardiac puncture, and plasma was stored at −20° C. until further analysis. Plasma glucose was measured using the Analox GM7 (Analox Instruments USA, Lunenburg, Mass.), Triglyceride (TG) and free fatty acid (FFA) were measured using enzymatic, colorimetric assays (Sigma, St. Louis, Mo. and Wako Chemicals USA, Richmond, Va., respectively), and insulin was measured using a RIA kit (Linco Research, St. Charles, Mo.). Plasma leptin and adiponectin were measured using specific enzyme-immunoassay kits (ELISA) designed for quantitative determination of mouse plasma samples (Alpco Diagnostics, Salem, N.H.).

### Indirect Calorimetry and Physical Activity Measurements

An open-ended indirect calorimetry system was used to measure oxygen consumption (O₂) and carbon dioxide (CO₂) production in mice for the calculation of metabolic rate and respiratory quotient (RQ). Animals were placed in 4 metabolic chambers for measurements taken over three days with free access to food and water. The differential O₂ and CO₂ concentrations, flow rate, RQ and metabolic rate (Weir equation) are calculated and stored in a computer configured with data acquisition hardware (Analogic, Wakefield, Mass.) and software (Labtech, Wilmington, Mass.). Average daily food intake was also determined in the indirect calorimetry settings. In addition, measurements of physical activity were carried out using the Columbus Instruments Opto M3, a multi-channel activity monitor that utilizes infrared beams to monitor an animal's movement in the X, Y and Z axis. The total physical activity during a three day calorimetry experiment was determined by adding all the ambulatory counts in X direction.

### Quantitative Real Time PCR

Different regions of the brain including cortex, brain stem, cerebellum, hypothalamus, and hippocampus were collected from 6 mo anesthetized mice, flash frozen and stored at −80° C. until processing. Total RNA was extracted from homogenized tissue using both TRIZOL reagent (Invitrogen) and RNeasy Mini Kit (Qiagen). Reverse transcription was performed using one μg total RNA with iScript cDNA systhesis kit (Bio-Rad). Quantitative PCR was performed using primer sets for genes of interest and three reference genes and iQ Supermix or iQ SYBR Supermix (Bio-Rad) following the manufacturer's protocols. Reactions were run in duplicate on an iQ5 Real-Time PCR Detection System (Bio-Rad) along with a no-template control per gene. The cycling conditions consisted of 3 min initial denaturation at 95° C. and 45 cycles at 95° C. for 10 sec and 55° C. for 30 seconds. Melt curve analysis was included for samples run with iQ SYBR Supermix. RNA expression data were normalized to levels of the reference genes Gapdh, ubiquitin C (Ubc), and actin-beta (Actb) using the comparative threshold cycle method.

### Lipidomic Analyses of Brain Tissues

Whole brain was extracted and frozen immediately in isopentene.

Fatty Acids (FAs). An Agilent 1100-LC system (Agilent Technologies, Palo Alto, Calif.) coupled to a 1946A-MS detector equipped with an electrospray ionization (ESI) interface (Agilent Technologies) was used to analyze unesterified fatty acids. A reversed-phase XDB Eclipse C₁₈ column (50×4.6 mm i.d., 1.8 μm; Zorbax; Agilent Technologies) eluted with a linear gradient from 90% to 100% of A in B for 2.5 min at a flow rate of 1.5 ml/min with column temperature at 40° C. was used. Mobile phase A consisted of methanol containing 0.25% acetic acid and 5 mM ammonium acetate; mobile phase B consisted of water containing 0.25% acetic acid and 5 mM ammonium acetate. ESI was in the negative mode, capillary voltage was set at 4 kV, and fragmentor voltage was 100 V. N₂ was used as drying gas at a flow rate of 13 liters/min and a temperature of 350° C. Nebulizer pressure was set at 60 psi. Commercially available fatty acids was used as reference standards, monitoring deprotonated molecular ions [M-H]⁻ in the SIM mode. HDA (m/z=269) was the internal standard.

Monoacylglycerols (MAGs). MAGs were separated on a XDB Eclipse C₁₈ column (50×4.6 mm i.d., 1.8 μm; Zorbax; Agilent Technologies). They were eluted with a gradient of methanol in water (from 85% to 90% methanol in 2.0 min and 90% to 100% in 3.0 min) at a flow rate of 1.5 ml/min. Column temperature was kept at 40° C. MS detection was in the positive ionization mode, capillary voltage was set at 3 kV, and fragmentor voltage was 120 V. N₂ was used as drying gas at a flow rate of 13 liters/min and a temperature of 350° C. Nebulizer pressure was set at 60 psi. Commercial MAGs were used as reference standards. For quantification purposes, we monitored the Na⁺ adducts of the molecular ions [M+Na]⁺ in the selected ion-monitoring (SIM) mode, using HDG (m/z 367) as an internal standard.

Diacylglycerols (DAGs). An Agilent 1100-LC system coupled to a MS detector Ion-Trap XCT interfaced with ESI (Agilent Technologies) was used. DAGs were separated using a reversed-phase Poroshell 300SB C₁₈ column (2.1×75 mm i.d., 5 μm; Agilent). A linear gradient from 60% to 100% of mobile phase A in B in 5 min at a flow rate of 1.0 mL/min with column temperature at 30° C. was applied. Mobile phase A consisted of methanol containing 0.25% acetic acid, 5 mM ammonium acetate; mobile phase B consisted of water containing 0.25% acetic acid, 5 mM ammonium acetate. The capillary voltage was set at 4.0 kV and skimmer voltage at 40 V. N₂ was used as drying gas at a flow rate of 12 liters/min, temperature at 350° C., and nebulizer pressure at 80 psi. Helium was used as collision gas, and fragmentation amplitude was set at 1.2 V. MS detection was in the positive ionization mode. DAGs were identified based on their retention times and MS³ properties, using synthetic standards as references. Multiple reaction monitoring was used to acquire full-scan tandem MS spectra of selected DAG ions. Extracted ion chromatograms were used to quantify each DAG by selecting appropriate precursor>product ions. DHDG (m/z 619.8>349.5) was the internal standard.

Triacylglycerols (TAGs). TAGs were separated on a Poroshell 300 SBC18 column (2.1×75 mm i.d., 5 μm, Agilent Technologies) at 50° C. A linear gradient of methanol in water containing 5 mM ammonium acetate and 0.25% acetic acid (from 85% to 100% of methanol in 4 min) was applied at a flow rate of 1 mL/min. Capillary voltage was 4.5 kV, skim1−40 V, and capillary exit −151 V. N2 was used as drying gas at a flow rate of 10 liters/min, temperature of 350° C. and nebulizer pressure of 60 PSI. Helium was used as collision gas. Lipids were identified by comparison with retention times and MSn fragmentation patterns of authentic standards. Detection was set in positive mode and TAGs were detected as NH₄⁺ adduct of the molecular ions. Full-scan MSn spectra of selected lipid precursor-ions was acquired and monitored characteristic product-ions using trinonadecenoin (m/z 944.8>631.6) as internal standard.

### Statistical Analyses

Results are presented as mean±SE. T-tests were performed using SigmaStat 2.03 (San Rafel, Calif.). A p<0.05 was considered significant.

### Results

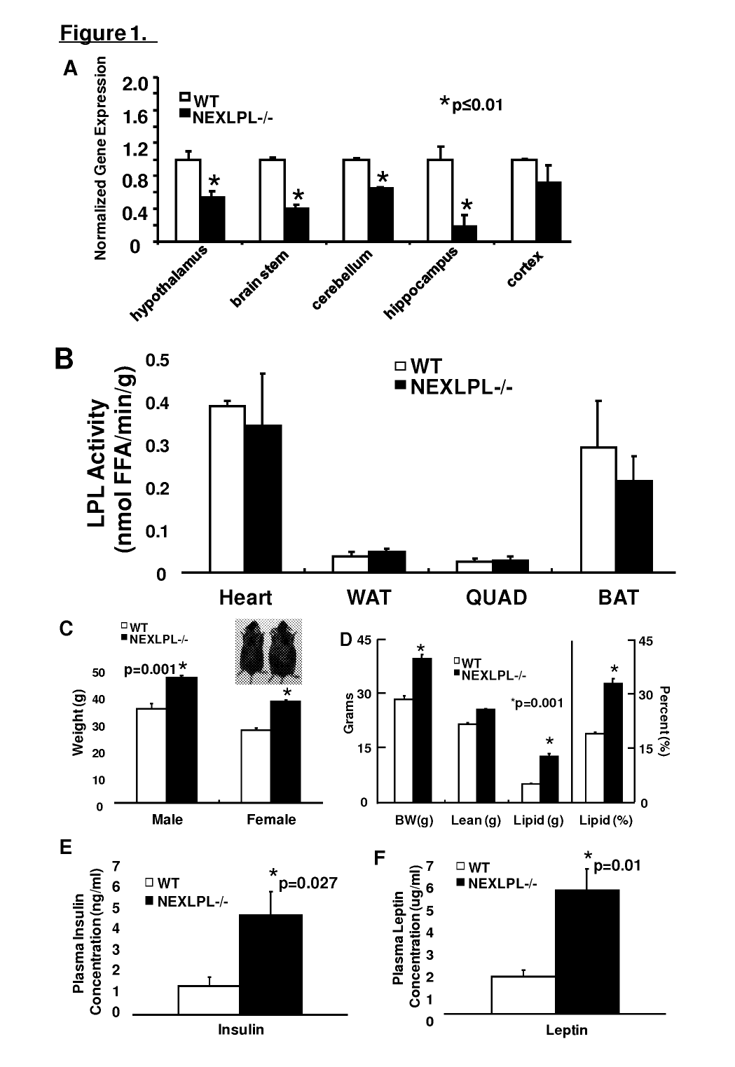

The crelox-P recombination system and the recently available NEX-Cre mice was used to successfully generate a neuron-specific LPL deficient mouse model (i.e., NEXLPL−/−). NEX, a gene that encodes a neuronal specific basic helix-loop-helix (bHLH) protein, allows the cre activity in NEX-Cre mice to be expressed in the neocortex, hippocampus, mid- and hindbrain regions, pyramidal neurons and dentate gyrus mossy and granule cells in the dorsal telencephalon, but not in regions containing proliferating neural precursors of the ventricular zone, interneurons, oligodendrocytes, or astrocytes. In NEXLPL−/− mice, LPL mRNA was substantially unchanged in the cortex, but reduced 40% to 60% in the brain stem, cerebellum and hypothalamus, and reduced by 80% in the hippocampal region (FIG. 1A). Additionally, NEX cre recombinase deletion of the floxed LPL gene did not significantly change the expression of LPL in other organs/tissues (FIG. 1B).

At 6 mo of age, highly significant weight increases were observed in homozygous male and female NEXLPL−/− (KO) mice that were fed chow (FIG. 1C) (29% increase in male, 38% increase in female) (p=0.001). Although some increase in lean body mass was seen (consistent with human obesity), most of the weight increase was due to increases in fat mass with the relative amount of fat increased from 19% in WT mice to 34% for NEXLPL−/− mice (FIG. 1D). Visual inspection of the NEXLPL−/− mice demonstrated increases in the abdominal and perigonadal WAT areas, and the suprascapular BAT fat pads. Other organs/tissues in the NEXLPL−/− mice appeared to be anatomically normal. Measurement of metabolic parameters showed that after 4 hr of fasting plasma insulin (FIG. 1E) and leptin (FIG. 1F) levels were elevated in 6 mo old NEXLPL−/− mice, while fasting plasma glucose, TG, FFA, and adiponectin levels remained normal. Table 1.

TABLE 1
Plasma metabolites and biomarkers for NEXLPL mice at 6 mo
(n = 4 for each group of mice)

| | Insulin | FFA | Glucose | TG | Leptin | Adiponectin |
|---|---|---|---|---|---|---|
| | ng/ml | mmol/l | mg/dl | mg/dl | ug/ml | pg/ml |
| WT | 1.4 ± 0.4 | 0.28 ± 0.06 | 180 ± 10 | 35 ± 6 | 1.7 ± 0.3 | 75 ± 8 |
| NEXLPL+/− | 1.2 ± 0.4 | 0.40 ± 0.06 | 162 ± 19 | 50 ± 5 | 1.0 ± 0.6 | 77 ± 4 |
| NEXLPL−/− | *4.8 ± 1.1 | 0.59 ± 0.16 | 160 ± 18 | 30 ± 9 | *5.6 ± 1.0 | 87 ± 7 |

### Reductions in Metabolic Rate and Physical Activity Sustained the Weight Gain in Obese NEXLPL−/− Mice

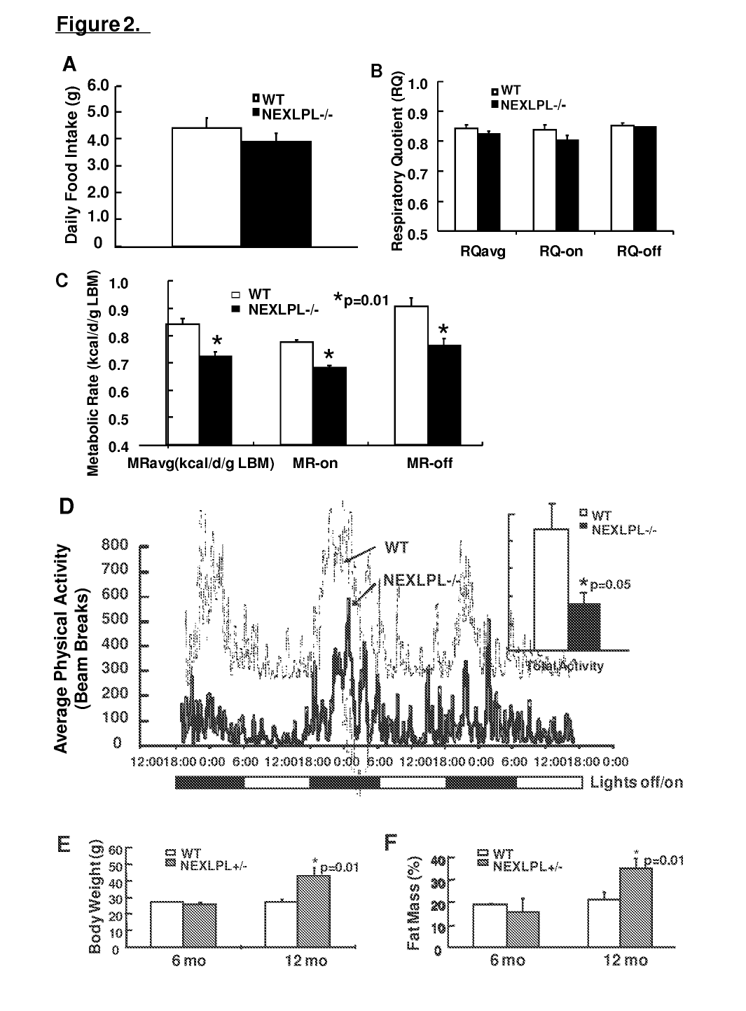

Food intake in NEXLPL−/− and WT mice was measured weekly in individualized cages under normal housing conditions between the ages of 6 and 12 mo. Average daily food intake during this interval was essentially the same for NEXLPL−/− and WT mice (FIG. 2A). Indirect calorimetry was next used to measure energy balance in 6 mo old NEXLPL−/− and WT mice. In the calorimeter, average respiratory quotient (RQ) over the 3 day period (during both lights-off and lights-on period) did not differ between NEXLPL−/− and WT mice (FIG. 2B). However, the average metabolic rate (MR) was significantly lower in NEXLPL−/− mice during both the lights-off and lights-on period (FIG. 2C), and the total number of beam breaks showed that NEXLPL−/− mice displayed a substantial reduction in physical activity (FIG. 2D insert). NEXLPL−/− mice also displayed a substantially sluggish response to the change of environmental conditions during the first day in the calorimeter, indicating a possible adaptive behavioral defect as well (FIG. 2D, first light off/on period).

### Modifications in Both Energy Intake and Energy Expenditure Contribute to the Development of Obesity in Heterozygous NEXLPL+/− Mice

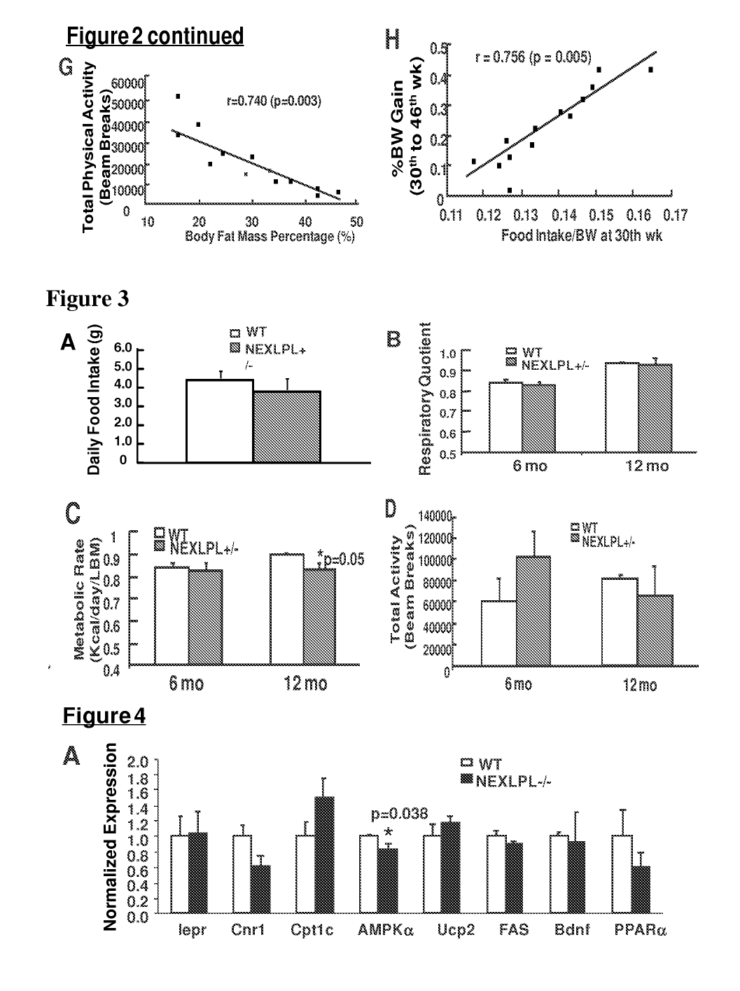

Heterozygous mice (NEXLPL+/−) showed no significant differences in weight compared to WT mice at 6 mo, but variably developed obesity as they aged (FIG. 2E). The extra weight gain at 12 mo in NEXLPL+/− mice, similar to the amount of weight gain in NEXLPL−/− mice at 6 mo, was mostly due to an increase in fat mass (FIG. 2F). The plasma metabolic parameters (Table 1) and food intake (FIG. 3A) for NEXLPL+/− mice were all similar to WT mice at 6 mo. RQ (FIG. 3B), and metabolic rate (FIG. 3C) were also not different for NEXLPL+/−at 6 mo, but a slight increase in physical activity was observed (FIG. 3D). At 12 mo, obese NEXLPL+/− mice maintained the same RQ compared to WT mice (FIG. 3B), but showed some reduction in metabolic rate (FIG. 3C) and variable reductions in physical activity (FIG. 3D). In fact, at 12 mo the energy expended in physical activity was inversely correlated to the level of obesity (fat mass percentage) in NEXLPL+/− mice (FIG. 2G).

During the development of obesity in NEXLPL+/− mice, a short period of increased food intake was observed between the 29^{th} wk and 32^{nd} wk. As these NEXLPL+/− mice started to gain extra body weight around 36 wk, food intake returned to the level of WT mice and remained low as the mice aged and further accumulated fat mass. The later development of obesity in NEXLPL+/− mice was predicted by the earlier increase in food intake at the 30^{th} wk (r=0.756, p<0.005, FIG. 2H).

### Alterations of Gene Expressions in Hypothalamus o NEXLPL−/− and NEXLPL+/−Mice.

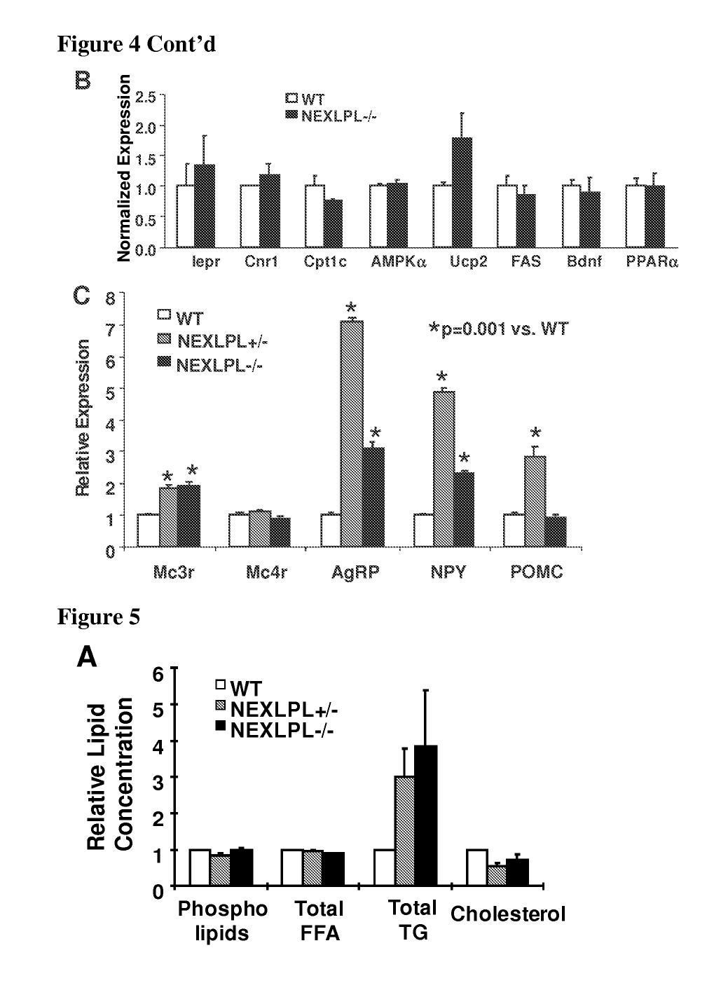

To determine mechanisms by which reductions in neuron-specific LPL gene expression might modify energy balance and body weight, mRNA levels of a selected group of genes known to be involved in the CNS glucose sensing, energy balance, and body weight regulation were examined in the hypothalamus (FIG. 4A) and hippocampus (FIG. 4B) in 6 mo old NEXLPL−/− mice. The mRNA levels of the leptin receptor (lepr), endocannabnoid receptor (CB1/Cnr1), carnitine palmitoyltransferase 1c (Cpt1c), AMP-activated protein kinase alpha 2 (AMPKcc2), uncoupling protein 2 (Ucp2), fatty acid synthase (FAS), brain derived neuronal factor (Bdnf), peroxisome proliferator activated receptor alpha (PPARα) were not changed in the hypothalamic or hippocampal regions of the brain, except for a modest reduction of AMPKcc2 (17%) in the hypothalamus that was statistically significant (FIG. 4A). In contrast, significant changes were observed in the expression of several genes of the melanocortin-4/3 receptor (Mc4/3r) pathway in the hypothalamus that are known to play a pivotal role in maintaining energy homeostasis. The mRNA levels of two of the orexigenic neuropeptides AgRP and NPY genes were found to be increased in the hypothalamus of NEXLPL−/− mice at 6 mo of age (3.1 fold for AgRP and 2.3 fold for NPY), and Mc3r but not Mc4r mRNA was increased 93% in NEXLPL−/− mice (FIG. 4C). The level of mRNA for another orexigenic neuropeptide, melanin-concentrating hormone (MCH), was essentially the same between NEXLPL−/− and control mice (FIG. 4C). No significant change in POMC gene expression at 6 mo was seen in NEXLPL−/− mice. The Mc4/3r related pathway of gene expression in NEXLPL+/− mice at 6 mo. was further examined. Surprisingly and unexpectedly, AgRP and NPY gene expression were increased even more in the 6 mo old NEXLPL+/− mice (7.1 fold for NEXLPL+/−vs. 3.1 fold for NEXLPL−/− for AgRP; 4.8 fold for NEXLPL+/−vs. 2.3 fold for NEXLPL−/− for NPY) (FIG. 4C). Mc3r mRNA expression in NEXLPL+/− mice was also increased to the similar levels as in NEXLPL−/− mice.

### Alterations in Brain Lipid Metabolism in NEXLPL−/− and NEXLPL+/− Mice

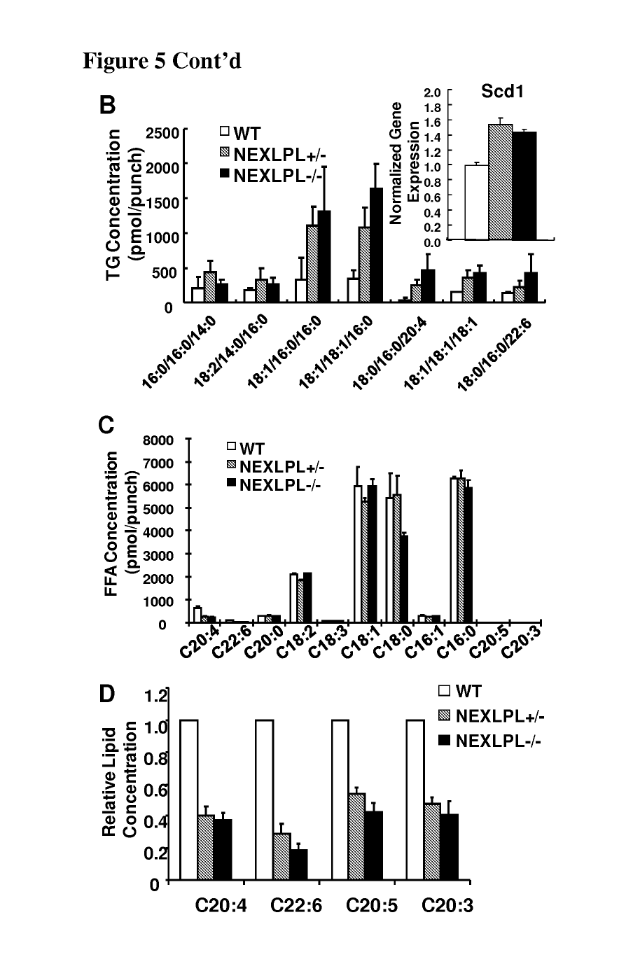

To assess the effect of decreases in neuronal LPL gene expression to the overall brain lipid profile, lipidomics analyses were conducted in various brain regions of 12 mo NEXLPL−/− and NEXLPL+/− mice. Total brain lipid levels were substantially altered in multiple brain regions including the hypothalamus, frontal cortex, thalamus and hippocampus. Particularly striking was the significant increase of TGs in the hypothalamus, and in both NEXLPL−/− and NEXLPL+/− mice (FIG. 5A). Total phospholipids and total FFAs remained the same, while total cholesterol was decreased in the hypothalamus. When analyzing the different species of TG, the increase in total TG appeared to be mostly due to the increase of monounsaturated species (FIG. 5B). Consistent with this observation, the expression of stearoyl-CoA desaturase-1 (SCD-1) was significantly increased in the hypothalamus of both NEXLPL−/− and NEXLPL+/− mice (FIG. 5B insert). These MUFAs however, were changed slightly in the FFA pool (FIG. 5C). Instead, the concentrations of some of the relatively less abundant FFA species, such as 20:4n-6 (omega-6), and 22:6n-3, 20:3n-3, 20:5n-3 (omega-3) were substantially lower in both NEXLPL−/− and NEXLPL+/− mice. (FIG. 5D). When comparing the relative concentration of 20:4n-6 in TG, diacylglycerol (DAG), monoacylglycerol (MAG), and FFA pools (FIG. 5E-5H), a clear contrast was observed as the level of 20:4n-6 was noted, i.e., increased in the TG pool, but decreased in the DAG, MAG, and FFA pools in both the NEXLPL−/− and NEXLPL+/− mice. These data indicate a defect in hydrolyzing TGs to make DAG, MAG, and FFA in the hypothalamus when LPL is deficient and the defect seems to be specific for long-chain PUFAs, not for various MUFAs and saturated lipids. To further examine related mechanisms, the expression of two of the enzymes that are involved in the biosythesis of these long-chain PUFAs were examined in the hypothalamus (FIG. 5I) and liver (FIG. 5J) in NEXLPL−/− and NEXLPL+/− mice. The expression level of A-5 desaturase (Fads2) was increased 20-25% in the hypothalamus in both NEXLPL−/− and NEXLPL+/− mice, while the level of A-6 desaturase was increased 76% in the hypothalamus of NEXLPL−/− mice. No significant change in the expression of both desaturases was seen in the liver of NEXLPL mice.

### Discussion

The present inventors have shown that LPL was expressed and synthesized in neurons in different brain regions including the cerebellum, cerebral cortex, hypothalamus, pituitary, and hippocampus. LPL is an important enzyme that contributes in a pronounced way to normal TG-rich lipoprotein metabolism, tissue-specific substrate delivery and utilization, and to the many aspects of obesity and other metabolic disorders that relate to energy balance, insulin action, and body weight regulation; however, these roles have all been attributed to LPL in peripheral tissues. Due to the increasing evidence that lipids in the CNS are important regulators of energy balance and body weight/composition, the present inventors have developed a physiologically relevant model to evaluate the in vivo function of LPL in the brain, and examined whether TG-rich lipoproteins are sensed in the brain.

The present inventors have shown herein that the neuron-specific LPL deficient mice developed obesity at a younger age (before 6 mo) in both male and female mice on a standard chow diet, and the degree of obesity was the most severe among all existing LPL deficient mouse models. This mouse shows a previously unidentified important function of LPL in CNS neurons, and the potential role of TG-rich lipoprotein sensing as a mechanism of CNS regulation of energy balance and body weight.

Detailed metabolic characterization of 6 mo old obese NEXLPL−/− mice showed that reduced energy expenditure (metabolic rate), mostly in the form of reduced physical activity, sustained the continuous fat accumulation in these already obese mice. Further characterization of heterozygous NEXLPL+/− mice between 6 mo and 12 mo indicated a two-step time course for obesity development: a period of hyperphagia before the onset of obesity, and a subsequent period of marked reduction in metabolic rate and physical activity that contributed to the further development of obesity. The fact that 12 mo old obese NEXLPL+/− mice displayed almost identical phenotypes as the 6 mo old obese NEXLPL−/− mice suggests that the development of obesity in NEXLPL−/− mice followed the similar path but with a much faster pace.

With the modifications of both energy intake and energy expenditure in NEXLPL mice, it was shown that expression of the orexigenic neuropeptides AgRP and NPY in both NEXLPL−/− and NEXLPL+/− mice at 6 mo. increased significantly. It has been shown that up-regulation of AgRP can increase energy intake as well as reduce energy expenditure. Such observation explains why food intake and energy expenditure are both modified in NEXLPL mice. It is noted that the food intake and energy expenditure are not modified in NEXLPL+/− mice at the same time. The increase in food intake appeared to be more transient (over a period of 2-3 weeks) but preceded the actual weight and fat mass gain. However, reductions in physical activity/metabolic rate were more persistent, accompanying and sustaining the continuing weight and fat mass gain.

Another surprising and unexpected observation is that the increase of AgRP/NPY gene expression was more in NEXLPL+/− mice than in NEXLPL−/− at 6 mo. Although at this age NEXLPL+/− mice displayed almost identical phenotypes as WT mice, the present inventors have shown that hyperphagia soon follows (starting as early as the 29^{th} week for some NEXLPL+/− mice), with subsequent reductions in physical activity and metabolic rate (starting as early as the 36^{th} week for some NEXLPL+/− mice). Considering NEXLPL−/− mice were already obese at this age and displayed increased AgRP and NPY gene expression, it is expected that the much higher levels of AgRP and NPY mRNA in the hypothalamus of normal weight 6 mo old NEXLPL+/− mice would predict the increase in food intake to follow. In some instances, the slight increase in physical activity of NEXLPL+/−at 6 mo can be explained by the up-regulation of the POMC gene (2.8 fold increase), a potential compensatory response to the up-regulation of AgRP/NPY expressing neurons. Compared to NEXLPL+/− mice, the more limited increase in AgRP/NPY gene expression with a normal level of POMC gene expression in already obese 6 mo old NEXLPL−/− mice could be the net result of other compensatory responses to obesity.

Besides the significant increases in AgRP/NPY gene expression, there was also a ˜90% increase expression of Mc3r but no change in expression of Mc4r in both NEXLPL−/− and NEXLPL+/− mice. AgRP and NPY are orexigenic neuropeptides that act as natural antagonists at the level of both Mc3 and Mc4 receptors. The increased expression of Mc3r may be a compensatory effect of the substantially increased gene expression of AgRP/NPY in the hypothalamus. However, the Mc4r level remained unchanged in NEXLPL−/− mice. Mc3r is often co-localized with Mc4r, and in some cases might share redundant functions as Mc4r, but evidence shows that its function in obesity, cachexia, and related feeding behaviors might involve different signaling pathways/regulatory mechanisms. Mouse model disclosed herein can be a tool in which to uncover the complex role of Mc3r in the regulation of energy balance and body weight.

The reduction of LPL gene expression in neurons appeared to selectively activate the AgRP/NPY neuronal activities without a significant effect on POMC neurons. The differential regulation of the melanocortin receptor system is consistent with the fact that Mc4r plays a more important role in glucose sensing and regulation of glucose metabolism via POMC neurons, and there were no changes in plasma glucose in mice disclosed herein.

Considering the key role of LPL in peripheral tissues as the rate limiting enzyme for the uptake of fatty acids from the hydrolysis of circulating TG-rich lipoproteins for lipid storage and/or oxidation, it appears that LPL plays at least some role in regulating brain lipid metabolism. The significant increase of total TG in all four brain areas was surprising and unexpected. In peripheral tissues, knocking down LPL in skeletal muscle and heart resulted in decreases in TG content in these tissues. However, considering the particular membrane structure in the brain cells and the requirement of active transport of lipid molecules across the blood brain barriers, it is believed that the TG accumulation in the hypothalamus as well as in other regions of the brain is the result of TG-rich lipoprotein being inadequately hydrolyzed due to LPL deficiency and accumulation in non-neuronal cells in the brain. This in turn results in the inability to utilize lipoprotein-derived TGs and incorporate resultant fatty acids into the lipid pools in neurons. Importantly, only the long chain PUFAs seemed to be significantly deficient in this LPL-dependent pathway, while the total FFA, MUFAs and saturated FFAs remained substantially the same. This shows that LPL plays a regulatory role in the provision of PUFAs to the murine brain.

The dietary essential PUFAs, especially docosahexaenoic acid [DHA; 22:6(n-3)] has been indicated as a critical contributor to cell structure and function in the nervous system, and deficits in DHA abundance are associated with cognitive decline during aging and in neurodegenerative disease. Furthermore, PUFAs have been implicated in the prevention of various human diseases including obesity, diabetes, coronary heart disease, stroke, and a number of inflammatory diseases. PUFAs have been proposed to function by altering membrane lipid composition, cellular metabolism, signal transduction, and the regulation of gene expression. However, most of these studies have been conducted in peripheral tissues, e.g. the regulation of many genes of lipid and lipoprotein metabolism in the liver. The decreased PUFA levels and increased expression of AgRP/NPY in the hypothalamus of NEXLPL mice of the present invention show that PUFAs function in the brain to regulate the expression of key neuropeptides that are important in the CNS regulation of energy balance and body weight via a LPL-dependent mechanism.

Neuron-specific reductions in LPL gene expression in mice result in severe obesity. This phenotype appears to be a consequence of a period of increased food intake followed by more sustained reductions in physical activity and metabolic rate. This progressive change in energy balance is most likely due to LPL-dependent decreased PUFA levels, and increased AgRP/NPY gene expression in the hypothalamus. Just as important as the gate-keeping role of LPL in determining the lipid partitioning in tissues such as adipose tissue and muscle, LPL in the murine brain appears to play a key role in energy balance and body weight regulation, by the generation of TG-rich lipoprotein-derived PUFAs in specific groups of hypothalamic neurons. The present invention provides CNS pathway that regulates energy balance and body weight.

### Example 2

This example provides a complimentary series of experiments using a model of brain-specific LPL deficiency and obesity. This model allows examination of how LPL in the brain contributes to energy balance and body weight regulation. One of the studies is to characterize the development of obesity in mice with neuron-specific deletion of lipoprotein lipase (NEXLPL−/− mice). It is believed that obesity in NEXLPL−/− mice is secondary to early increases in energy intake and then maintained by decreases in energy expenditure.

### Localization of CNS LPL Gene Expression

Regionalization of LPL deficiency in the CNS of NEXLPL−/− mice was determined by measurements of heparin releasable LPL activity and LPL mRNA by RT-PCR in several CNS regions. To further identify brain regions where the NEX-cre activity has reduced the expression of LPL, in situ hybridization is used to examine distinct regions of the brain and spinal cord that normally have NEX gene expression. These areas include the telencephalon (olfactory bulb, neocortex, hippocampus, amygdala), diencephalon (hypothalamus, thalamus), midbrain, and hindbrain (medulla, pons, cerebellum).

### In Situ Hybridization

A LPL riboprobe has been used to identify regions of the CNS in which LPL is expressed. Various riboprobes can be used to identify the regions. Alternatively, RP_{—}071204_{—}02_D12 can be used. This riboprobe is a murine sequence that has previously been used successfully to localize LPL mRNA in mouse brain (http://mouse.brain-map.org/brain/Lpl.html?ispopup=1). Briefly, sections of the brain and spinal cord is prepared from 3 months old female mice (slightly more pronounced phenotype than male mice at 6 months), pre-washed, and baked overnight at 200° C. After dehydration the sections is pre-hybridized at 42° C. for 2 hr. The pre-hybridization solution is then tapped off with a hybridization mixture of 200K dpm/60 μL of either sense or anti-sense LPL riboprobe and 10 mM dithiothreitol (DTT). The resulting mixture is incubated at 42° C. for 16 hr. Sense and anti-sense 3%-labeled LPL riboprobes are generated from RP_{—}071204_{—}02_D12 in a PGEM-7 vector using either T7 or SP6 RNA polymerases. Sections are then washed in 4×SSC with 10 mM DTT at 23° C. for 15 mM, then in 2×SSC for 30 mM, then 0.1% SSC in 0.1% SDS at 50° C. for 15 mM. Sections are then dehydrated, dipped in Kodak NTB-2 emulsion, and stored at 4° C. in light-tight boxes for 7 days before being developed, counterstained with hematoxylin, mounted, and examined by light or darkfield microscopy.

A total of 20 mice (10 of each phenotype) is used for this experiment.

### Phenotypic Development

As demonstrated in Example 1, NEXLPL−/− mice are quite obese at 6 months, and early experiments from the calorimeter indicate that the predominant alteration in energy balance is in energy expenditure, particularly during the dark cycle. The ontogeny of the phenotype is examined in this novel model of obesity to provide additional insights into the mechanism of obesity development, e.g., to determine if the mechanism involves energy expenditure side of the energy balance equation only or whether it also increases energy intake, particularly in the earlier phases of phenotypic development.

Characterization of the development of obesity in NEXLPL−/− mice requires detailed data gathered at regular intervals for 1 yr. The number of mice (male+female) examined at each time point for control and NEXLPL−/− mice (2 groups) for each determinant is listed in Table 1. A total number of 576 mice (144 of each phenotype and gender) is used for these experiments.

TABLE 1

| | 6 wk | 12 wk | 18 wk | 26 wk | 39 wk | 52 wk |
|---|---|---|---|---|---|---|
| Individualized | | | | | | |
| Cages | | | | | | |
| Body Weight | 24 × 2 | 24 × 2 | 24 × 2 | 24 × 2 | 24 × 2 | 24 × 2 |
| Food Intake | groups | groups | groups | groups | groups | groups |
| Body | | | | | | |
| Composition | | | | | | |
| Plasma and | | | | | | |
| Tissue | | | | | | |
| Measurements | | | | | | |
| Whole Room | | | | | | |
| Calorimetry | | | | | | |
| Food Intake | 24 × 2 | 24 × 2 | 24 × 2 | 24 × 2 | 24 × 2 | 24 × 2 |
| Energy | groups | groups | groups | groups | groups | groups |
| Expenditure | | | | | | |
| Energy Balance | | | | | | |
| CHO Balance | | | | | | |
| Fat Balance | | | | | | |
| Physical | | | | | | |
| Activity | | | | | | |

Whole room calorimetry is performed as described, for example, by Jensen et al., in J. Appl. Physiol., 2001, 90, 912-918 and J. Lipid Res., 2008, 49, 870-879, 2008. This is an open-ended indirect calorimetry system used to measure O₂ consumption and CO₂ production in mice with the option of examining energy balance at various ambient temperatures. Briefly, air from a common air source is pulled through four metabolic chambers (Metabowl), a blank cage, and a reference line. The differential measurements of O₂ and CO₂ compared with the reference line, a total 3.6×10⁵ measurements, are performed continuously every 10 min for each of the cages with the use of a paramagnetic O₂ analyzer and infrared CO₂ analyzer (Oxymat/Ultramat 6; Siemens, Roswell, Ga.). The analyzers are calibrated before each experiment with a primary standard of O₂ (0.950%) and CO₂ (0.770%), with the balance being composed of nitrogen (General Air, Denver, Colo.). Any small fluctuations in the differential readings are measured in the blank fifth cage and accounted for in the calculations of RQ and MR (Weir equation).

Physical activity during the period of calorimetry is monitored with the use of Opto-M3 Activity Meters (Columbus Instruments, Columbus, Ohio) customized to work with metabolic cages. Using infrared beams, activity is monitored in both the horizontal (x-axis) and vertical (z-axis) directions. For each of the axes, total and ambulatory (new) beam breaks are detected and analyzed. The activity monitors is validated for each of the cages by correlating the total beam breaks for 12.5 min with the MR measured over the same period.

Energy expenditure and oxidation of protein, CHO, and fat are determined from these measurements. Briefly daily rates of oxidation of protein, carbohydrate, and fat are determined for each 72 hr stay in the whole-room calorimeter. Protein oxidation is determined from 24-hr urinary nitrogen excretion (measured by using the Kjeldahl technique), and carbohydrate and fat oxidation are determined from the total energy expenditure and the non-protein RQ. Nutrient balance is calculated as the difference between intake and oxidation of each macronutrient over 72 hr.

### Plasma and Tissue Measurements

Plasma determinations (4 hr fasted and fed) include glucose, FFA, triglycerides, cholesterol, HDL cholesterol, insulin, leptin, adiponectin, and ghrelin. Tissues including the cerebral cortex, hippocampus, hypothalamus, cerebellum, upper spinal cord, heart, skeletal muscle, BAT and WAT are used to measure LPL activity. The triglyceride content of brain, heart, skeletal muscle and liver are also quantified.

### Results

The experiments to locate the sites in the brain where LPL is reduced or deleted by the NEX cre recombinase approach are limited in part for two reasons: (1) in situ is semi-quantitative so determination of only relative levels of expression within sites between WT and NEXLPL−/− are possible; and (2) sites where the LPL mRNA is reduced or deleted may not modify levels of enzyme protein in that same region. The fact that LPL protein has been localized by immunohistochemistry to endothelial surfaces throughout the brain, including regions where the LPL mRNA has not been identified confounds ones ability to extend the findings by in situ hybridization to the levels of the enzyme protein.

The time course of development of obesity in NEXLPL−/− mice is likely between 6 weeks and 3 months of life. During the 6 wk and 3 months interval, the energy intake increases, whereas Example 1 demonstrates reductions in energy expenditure. Because currently so little is known about LPL in the brain, it is interesting to discern whether or not preferential carbohydrate oxidation predicts the development of a positive energy balance in these mice as suggested by studies in pre-obese humans, rodents, and following weight reduction in reduced-obese humans. A number of additional experiments is of interest including whether high fat feeding exacerbates the phenotype and/or if pair-feeding modifies the phenotype.

### Example 3

This example determines if obesity in NEXLPL−/− mice is secondary to the deficiency of the LPL protein or the bioactive LPL.

The obesity in NEXLPL−/− mice is related to the absence of the LPL protein and the hydrolytic activity of the lipase. LPL deficiency in humans is rare (about 1/1,000,000) and the relationship to body fat deposition and/or obesity is not clear. Because most cases of LPL deficiency are compound heterozygotes with missence mutations in the LPL gene, a potential role of LPL independent of its bioactivity could mediate the effects of LPL on energy balance and body weight regulation in the CNS. To determines if obesity in NEXLPL−/− mice is secondary to the deficiency of the LPL protein or the bioactive LPL, two types of experiments are used.

### A. Cholesteryl Ester Ether Experiments

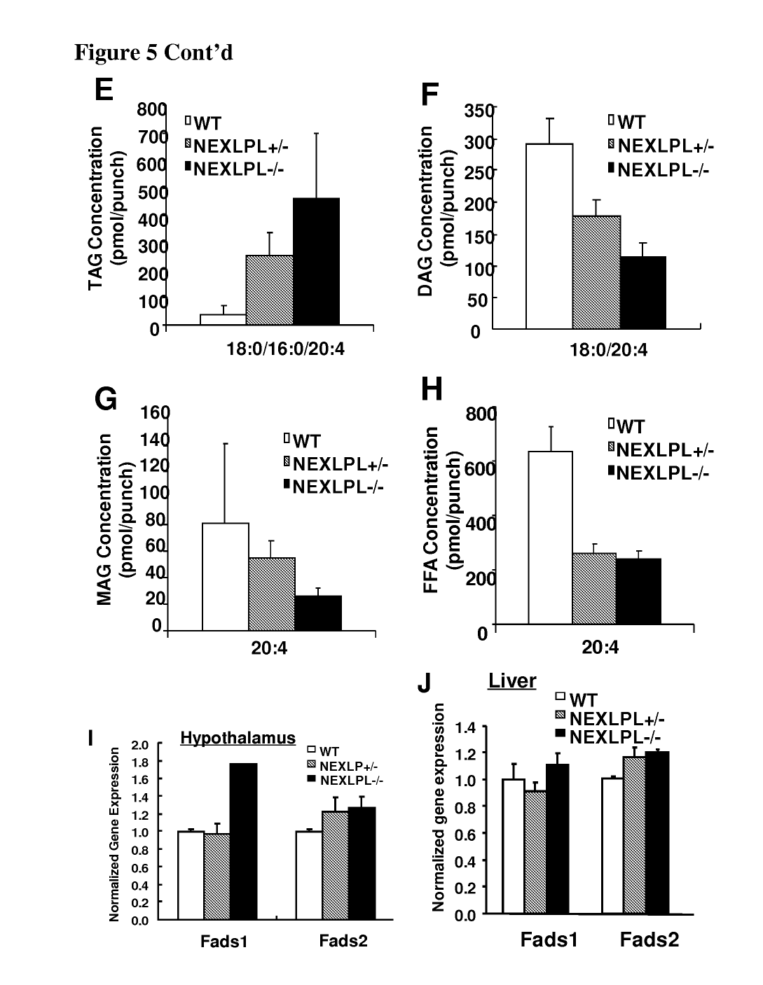

Although LPL hydrolyzes the TG core of TG-rich lipoproteins, cholesteryl ester ether (CE) is not a substrate for the enzyme. To determine whether Intralipid TG uptake in the brain is associated with uptake of non-hydrolyzed core lipids, double-labeled Intralipid with [³H]CE and [¹⁴C]TG is used as described by Augustus et al. in Am J Physiol Endocrinol Metab., 2003, 284, E331-E339. The ratio of brain uptake for the two labels characterizes the relative proportion of LPL-mediated neutral lipid uptake that is dependent on the hydrolytic function of the enzyme vs. the whole particle or remnant lipoprotein uptake. A diagram illustrating this experiment is shown in FIG. 6. Portrayed in this figure LPL is made in the neuron and transported to the capillary endothelium. Here the lipase binds TG-rich lipoproteins (CM, VLDL) that results in the hydrolysis of the core triglycerides releasing fatty acids and monoacylglycerol. These products of lipolysis are then transported through the blood brain barrier and enter brain cells (neurons and/or astrocytes). Alternatively, partially delipidated lipoproteins can be internalized by endocytotic mechanisms, a role clearly shown for active and inactive LPL in other tissues.

A total of 16 (8 of each phenotype) mice are used for this experiment.

Twenty percent Intralipid (Kabi Pharmacia, Clayton, N.C.) is diluted in sterile PBS to a final 5% concentration and labeled with 60 μCi [³H]triolein or 40 μCi [³H]cholesteryl oleoyl ether (CE) and 8 μCi [¹⁴C]triolein (Amersham Pharmacia Biotech), as described by van Bennekum et al. in J. Lipid Res., 1999, 40, 565-574. Labels are added to a small glass vial and slowly evaporated to dryness under N₂. Five hundred μL of a 5% solution of Intralipid is added to the small glass vial and sonicated 3 times for 20 sec each at a power level of 40 W to incorporate the triolein and CE into the emulsion. The resulting emulsion is stored at 4° C. before use in experiments. Double-labeled emulsion is injected i.v. into 8 fasting female WT and NEXLPL−/− mice at 3 months of age to assess whole particle uptake. Uptake is measured in the cerebral cortex, hippocampus, hypothalamus, and cerebellum at 10 min intervals for up to 30 min after injection. To determine if the cholesteryl ether component modifies the uptake of TG-rich lipoprotein-derived fatty acids, experiments is also carried out using the same substrate in the absence of CE. These studies also allow one to discern how the absence of LPL in selective brain regions modifies lipid uptake.

### B. Inactive LPL Knock-In Experiments

A construct (NEX-Cre) containing the coding sequence of cre recombinase with a 1.47 kb DNA fragment of the promoter region of NEX located immediately upstream of the coding region is obtained as described by Goebbels et al. in Genesis, 2006, 44, 611-621. This is the same construct that is used to create the NEX-Cre mice. Another construct containing an enzymatically inactive human LPL minigene driven by a muscle creatine kinase promoter (with point mutant Asp 156 to Asn) is obtained as described by Merkel et al. in Proc Natl Acad Sci USA, 1998, 95, 13841-13846. This point mutation produces a catalytically inactive LPL protein that is secreted normally from cells. The cDNA of 156N-LPL is cloned in frame to replace the cre recombinase in the NEX-cre construct, and a gene is created targeting construct that contains a catalytically inactive LPL minigene behind the NEX promoter sequence (NEXhLPLm).

All cell culture work and injections of recombinant ES cell clones into mouse embryos to generate chimeric mice are carried out using a similarly known process. In brief, 107 ES cells in 0.8 mL of PBS is transfected by electroporation with 50 μg of the targeting vector NEXhLPLm. Transfected ES cells are cultured on gelatinized dishes in the presence of 103 U/mL leukemia inhibitory factor (GIBCO), and selection with 300 μg/mL of G418 (GIBCO) is started 24 hr later. Correctly recombined ES cell clones are identified by PCR with designed primers. Microinjection of one ES cell clone is performed by standard procedures. Highly chimeric animals are crossed to C57BL/6J×CBAJ females (The Jackson Laboratory). Heterozygous offspring are interbred to generate homozygous NEXhLPLm mice.

NEXhLPLm mice are crossed with wild type mice or the NEXLPL−/− mice. Pups heterozygous for the transgene (NEXhLPLm+/−) as well as NEXLPL+/−are crossed with each other to create a brain-specific inactive LPL knock-in mouse line (NEXLPL-KI). The inactive LPL is driven by the same NEX promoter that was used to create the NEXLPL−/− mice; thus the catalytically inactive LPL are expressed in exactly the same regions in the brain where the endogenous LPL was deleted.

After NEXhLPLm mice have been created, a similar phenotypic characterization is performed as described in Example 2, e.g., body weight, food intake in individualized cages, body composition, and plasma and tissue metabolites. Only female mice are characterized in this experiment.

A total of 144 mice (72 KI and 72 controls) are used for this experiment.

### Results

The cholesteryl ester ether experiments demonstrate a non-hydrolytic role of LPL in the uptake of TG-rich lipoprotein lipid into the brain. Moreover, the uptake of TG-rich lipoprotein-derived fatty acids is only reduced in areas where LPL activity is reduced (predominantly the hippocampus). The second component of this example creates mice with a knock-in of the inactive lipase. One potential limitation of this strategy is that there may not be sufficiently high enough levels of expression of the inactive LPL to determine its role. When the result from cholesteryl ester ether experiments indicates a non-hydrolytic role of LPL in the brain but there is no improvement of the obesity phenotype in knock-in mice, a different strategy is used to create transgenic mice that overexpress inactive LPL. In this strategy, a 10-kb fragment of the NEX promoter is obtained, the promoter is tested for driving transgene expression, and transgenic mice are created that overexpress inactive LPL using the more traditional zygote injection technique. A different line of NEXhLPLm mice is then created to have brain-specific overexpression of the inactive LPL. However, it is believed that in some instances both the hydrolytic and non-hydrolytic functions of LPL are required for LPL to regulate body weight and energy balance in the brain.

### Example 4

This example determines the CNS regions in which the deficiency of LPL leads to obesity in mice.

The predominant brain region that is responsible for the obesity in NEXLPL−/− mice is the hippocampus. LPL mRNA is abundant in the hippocampus but the importance of this brain region to energy balance has not been determined. It is possible that the deletion of LPL in other brain regions, e.g., hypothalamus, may fit better with the obesity phenotype that follows deletion of the enzyme protein. However, currently there is no data on how the CNS senses lipoproteins. It is possible that the hippocampus develops pathways of substrate recognition that provide signals to other brain regions about body weight/composition. The synaptic communication between neurons in the hypothalamus and the hippocampus provide a network of communication that can relate LPL to energy balance. It is possible that over periods of medium to long term nutrient exposure, this process of communication develops with training of LPL-related signaling. It is also possible that LPL in these regions controls lipid delivery in a manner that couple pathways of lipogenesis to oxidative metabolism and maintain energy balance. Results of this experiment provide information about the relative roles of LPL in the hippocampus and hypothalamus in regulating energy balance. Regional LPL Reconstitution Experiments

a. Hippocampus—Lentiviral human synapsin I LPL (hSYNhLPL)

b. Arcuate Nucleus—Lentiviral hSYNhLPL

### Generation of Recombinant Lentivirus Directing the Neuronal Expression of LPL

The human synapsin 1 promoter is used to express the active lipase in mice. This promoter is convenient for packaging because it is small and the specificity for neuronal expression is >95%. The lentiviral vector containing the human synapsin 1 promoter is available, and the gene product specifically targets neurons in mice. The LPL cDNA is cloned into this vector. The co-expression of GFP with LPL allows one to profile the successfully transfected neurons in the hippocampus and regions of the hypothalamus. Before proceeding with packaging this transgene into lentiviruses, the production of active LPL is first examined in NSH neuroblastoma cells.

The same lentiviral vector lacking the target gene LPL and the one in which the target gene LPL is replaced by an irrelevant LacZ are used as two negative controls. Using a standard protocol, the lentiviruses is produced by transfecting HEK293T with the target vector as well as the package plasmids. The lentiviruses is then purified, concentrated, and titrated using a p24 ELISA kit. The sole presence of GFP in the neurons around the injection sites is then confirmed.

Generation of a Mouse Model with Hippocampal and Hypothalamic Arcuate Nucleus Injections of the LPL Lentivirus

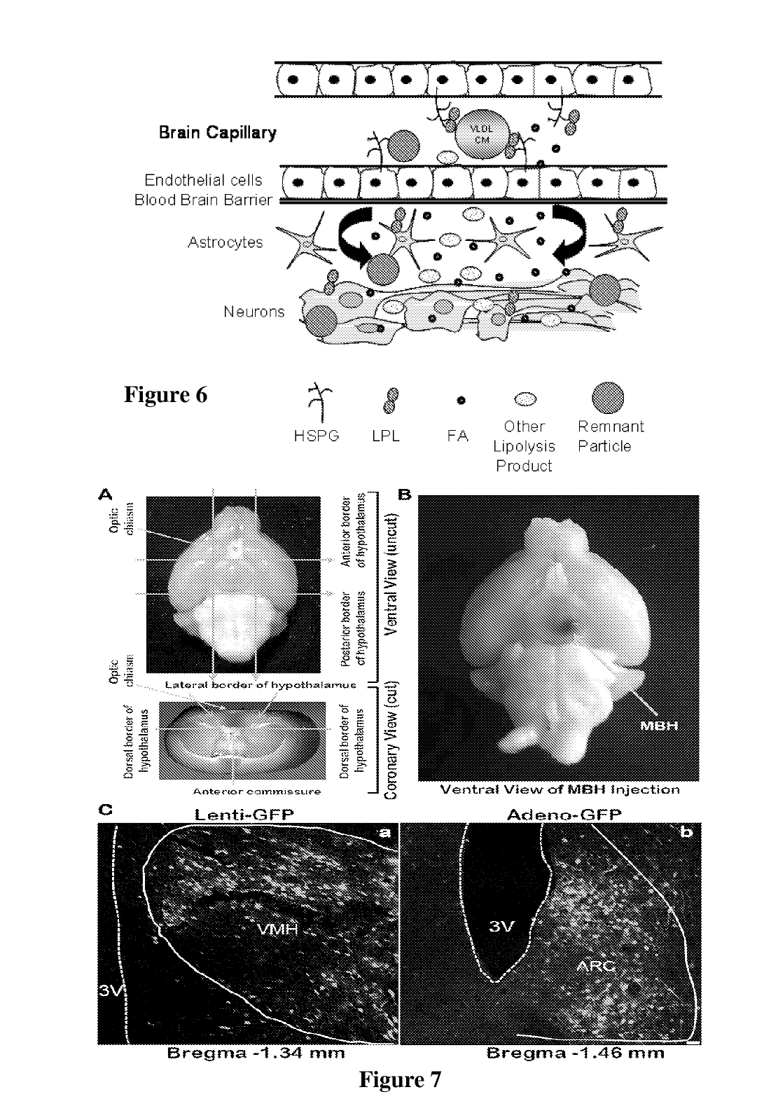

The lentivirus co-expressing GFP and LPL and the two controls are injected in separate experiments into the hippocampus and mediobasal hypothalamus (MBH) (FIG. 7A, 7B) of adult (3 mo old) female NEXLPL−/− mice. The intra-brain injections are directed using a stereotaxic table (Kopf Instruments). For the intra-MBH injections, the stereotaxic coordinates at 1.5 mm posterior to the bregma, ±0.3 mm lateral to midline, and 5.8 mm below the surface of the skull are targeted (FIG. 7B-D). For the intra-hippocampus injections, various coordinates are adjusted to target CA1-4 regions (FIG. 7B). Lentiviruses are then injected into these brain regions concentrations using a 26-gauge guide cannula and a 33-gauge injector (Plastics One) connected to a Hamilton Syringe and a syringe infusion pump. Hippocampal or hypothalamic over-expression of LPL results in local and neuron-specific production of LPL in the mice.

An example of the successful application of this technology targeting the hypothalamus using GFP-expressing lentiviruses (and adenoviruses) is shown in FIG. 7C. In this experiment, a GFP-expressing lentivirus (Lenti-GFP) (FIG. 7Ca) and a GFP-expressing adenovirus (Adeno-GFP) (FIG. 7Cb) were respectively delivered to the two specific regions in the hypothalamus of mice. The hippocampal and hypothalamic areas are injected with 3 μL of the lentiviral preparations (1.5×10⁷ TU) into the hippocampus or hypothalamus (using a 5 μL Hamilton syringe, 0.25 μL/min). Mice also received injections of the active lipase or vehicle into an irrelevant region, e.g., the cerebral cortex as a control. No impact on energy intake or body weight is expected from these injections. In addition, the inactive lipase used in Example 3 is packaged into lentivruses (Lenti-hSYNhLPLm) or vehicle alone. One month after all injections, mice are sacrificed and the brains immersion-fixed in 4% paraformaldehyde for subsequent immunohistochemical analysis.

### Verification of the Site Specificity of LPL Overexpression

For both experimental and WT mice as described above, the accuracy of injection-mediated gene delivery is evaluated at the end of the studies. To do this, the brains are sectioned through the hippocampus and hypothalamus to examine the distribution of GFP. Only animals with clear GFP fluorescence limited to both hippocampus, and ARC and VMH are included for the analysis. Mice are removed from the analysis based on the following criteria: GFP neurons are observed outside of the MBH or with less than 80% GFP neurons in the hippocampus, and the ARC or VMH. Some mice that show weak GFP expression along the cannula tract are accepted for the study. To calculate the percentage of GFP⁺ neurons, neuronal staining is performed for the sections using an anti-LPL antibody and a red fluorescence-coupled 2nd antibody, which are merged with GFP to show the distribution of GFP within all the MBH neurons.

### Phenotype Characterizations

After the Lenti hSYNhLPL construct injections, LPL mRNA and activity are measured in the selective brain regions and phenotypic characterization are carried out as outlined in the first set of basic measurements described in Example 2, e.g., body weight, food intake in individualized cages, body composition, calorimetry, and plasma measurements for up to one month. The number of mice used for experiments in Example 4 (n=288) is given as follows:

| | Hippocampus | Hypothalamus | Cortex |
|---|---|---|---|
| hSYNhLPL & | 24 × 2 groups | 24 × 2 groups | 24 × 2 groups |
| Control | | | |
| hSYNhLPLm & | 24 × 2 groups | 24 × 2 groups | 24 × 2 groups |
| Control | | | |

In addition to characterizations of the baseline levels of feeding and energy expenditure between the LPL and control virus-injected mice, their potential phenotypic differences in response to local administrations of TG-rich lipoproteins (Intralipid) through third ventricle injections is profiled. For comparison, other types of nutrient species such as glucose, fatty acids, and amino acids are similarly injected. In this procedure, the third ventricle immediately after the viral injections is implanted with a cannula. The third ventricle is targeted at the coordinate of about 1.8 mm posterior and about 5.0 mm ventral in the midline. After surgical recoveries, mice are placed into individualized cages and their food intake recorded over 72 hr after they receive intra-third ventricle injections of a nutrient or the vehicle control. The acute experiments of lipoprotein injections help to restrict any compound effects from long-term manipulations. The positive results from these acute experiments consolidate the hypothesized roles of LPL in the central control of energy balance and body weight homeostasis.

### Results

The injection of Lenti hSYNhLPL into the hippocampus decreases the weight gain in NEXLPL−/− mice. Currently, it is uncertain whether this effect occurs in the hypothalamus. This uncertainty is based on the fact that although LPL mRNA is reduced in the hypothalamus of NEXLPL−/− mice, enzyme activity is not. Although mice transgenic for the inactive lipase (NEXhLPLm) have a modest effect on the obesity phenotype (Example 3), we doubt that mice injected with Lenti hSYNhLPLm have this effect. This may simply be because of the modest effect expected in Example 3 and the lesser effects of injection of the inactive lipase gene construct in single areas of the brain.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A method of treating or preventing a clinical condition associated with lipoprotein lipase activity in the brain of a subject, said method comprising administering to the subject a composition comprising a compound that modulates lipoprotein lipase activity in the brain of the subject.

2. The method of claim 1, wherein the compound modulates the expression of lipoprotein lipase in the brain of the subject.

3. The method of claim 2, wherein the compound increases the expression of lipoprotein lipase in the brain of the subject.

4. The method of claim 1, wherein the clinical condition comprises obesity, Alzheimer's disease, dementia, memory loss, or depression.

5. The method of claim 1, wherein the compound increases the lipoprotein lipase activity in the brain of the subject.

6. The method of claim 1, wherein the compound is LPL or an analog or a derivative or a combination thereof.

7. The method of claim 6, wherein LPL is enzymatically active.

8. The method of claim 6, wherein LPL is enzymatically inactive.

9. The method of claim 1, wherein the compound upregulates the enzymatic activity of LPL in the brain.

10. The method of claim 1, wherein the compound downregulates the enzymatic activity of LPL in the brain.

11. The method of claim 1, wherein the compound upregulates the expression of LPL in the brain.

12. The method of claim 1, wherein the compound downregulates the expression of LPL in the brain.

13. A transgenic non-human mammal comprising neuronal specific LPL deficiency compared to the wild type of said non-human mammal.

14. The transgenic non-human mammal of claim 13, wherein said mammal is a rodent.

15. The transgenic non-human mammal of claim 14, wherein said rodent is a mouse.

16. The transgenic non-human mammal of claim 15, wherein the phenotype of said mouse is NEXLPL−/− or NEXLPL+/−.

17. A method for identifying a small molecule that is suitable for treating or preventing a clinical condition associated with decreased lipoprotein lipase activity in the brain of a subject, said method comprising:
administering the small molecule to a non-human mammal comprising neuronal specific LPL deficiency compared to the wild type of said non-human mammal; and
determining the level of neuronal specific LPL activity in the non-human mammal, wherein if the level of neuronal specific LPL activity in the non-human mammal is substantially similar to the level of neuronal specific LPL activity in the control, then it is indication that the small molecule is suitable for treating or preventing clinical condition associated with decreased lipoprotein lipase activity in the brain of a subject.

18. The method of claim 17, wherein the subject is human.

19. The method of claim 17, wherein the transgenic non-human mammal is a mouse.

20. The method of claim 19, wherein the phenotype of the mouse is NEXLPL−/− or NEXLPL+/−.

21. The method of claim 17, wherein the clinical condition comprises obesity, Alzheimer's disease, dementia, memory loss, or depression.
